(19) United States
(12) Patent Application Publication
(10) Pub. No.: US 2002/0035338 A1
(43) Pub. Date: Mar. 21, 2002

(54) Epileptic seizure detection and prediction by self-similar methods

(76) Inventors: Stephen P. Dear, Hummelstown, PA (US); Corey B. Hart, Middletown, PA (US)

Correspondence Address: SMITH, GAMBRELL & RUSSELL, LLP, SUITE 3100, PROMENADE II, 1230 PEACHTREE STREET, N.E., ATLANTA, GA, 30309-3592, US

(21) Appl. No.: 09/727,383

(22) Filed: Nov. 30, 2000

Related U.S. Application Data
(60) Provisional application No. 60/168,374, filed on Dec. 1, 1999.

(51) Int. Cl. A61B 5/04

(52) U.S. Cl. 600/544

(57) ABSTRACT

The present invention provides a system and method for the detection and prediction of epileptic seizures based on the calculation of a scaling exponent, scaling behavior, and fractal fraction values from a patient's brain wave activity data.

### BACKGROUND

After stroke, epilepsy is the most common neurological disorder, affecting approximately 20 to 40 million people worldwide. Diagnosis and treatment of epilepsy involves electroencephalographic (EEG) recordings of electrical potentials reflecting underlying brain activity. Epilepsy diagnosis and treatment would benefit if somehow EEG recordings could anticipate epileptic seizures. However, visual analysis of EEG epileptiform activity is not a reliable seizure predictor. Consequently, different approaches borrowing tools from nonlinear dynamic systems theory have been directed towards finding reflections of an internal brain “state.” The nonlinear approaches share a common underlying hypothesis, namely, that brain state changes before an epileptic seizure.

A number of studies have suggested that EEG signals recorded from the brain are chaotic (Babloyantz and Salazar 1985; Freeman and Skarda 1985; Rapp et al. 1985, 1988; Babloyantz and Destexhe 1986; Skarda and Freeman 1987; Basat et al. 1988; Nan and Jinghua 1988; Roschke and Basar 1988; Skinner et al. 1988; Basat and Bullock 1989). In such studies, chaotic behavior is indicated by graphing the EEG time-series amplitude against a time-delayed version of itself known as a phase plot. Trajectory lines in the phase plots continually returned to the same graph region, suggesting the presence of an underlying chaotic attractor in the EEGs. Using mathematical tools borrowed from nonlinear dynamic systems theory, the correlation dimension, D2, provides one estimate of the complexity of chaotic systems that give rise to chaotic attractors (Packard et al. 1980; Takens 1981; Farmer et al. 1983; Skinner 1991; Grassberger et al. 1993). Various forms of D2 computed from EEGs have been linked to underlying brain states such as waking, sleep, cognitive processing, etc. (Babloyantz and Destexhe 1986; Watt and Hameroff 1987, 1988; Pijn et al. 1991; Lehnertz and Elger 1995). These prior observations lead to the hypothesis that EEG correlation dimension is linked with epileptic seizures in that D2 estimates should decrease during the seizure due to increased neuronal synchronization (lasemidis and Sackellares 1996). This hypothesis was tested and verified in recent studies (Elger and Lehnertz 1998; Lehnertz and Elger 1998; Martinerie et al. 1998). These studies interestingly also demonstrated a noticeable decline in the computed D2 for a time interval preceding the seizure up to several minutes. While such results offer the possibility that D2 and related nonlinear dynamic dimensional estimates computed from EEG time-series can provide an interval of seizure prediction, D2 estimation has several shortcomings as a general applicable prediction method.

First, D2 based estimation includes several potential sources of error. Potential error sources include time-series nonstationaries, the presence of added noise, filter effects, too high or low digital sampling rates, and too short time series records (Mayer-Kress et al. 1986; Rapp 1994; Lerner 1996; Schiff 1998). These potential error sources have raised a question as to whether EEGs reflect low dimensional chaos or just colored noise based on the reanalysis of EEGs with improved D2 estimation algorithms or linear algorithms (Prichard et al. 1995; Palus 1996; Theile and Rapp 1996; Netoff et al. 1999). As a result, nonlinear dynamic algorithms may predict seizures, but for reasons that have nothing to do with nonlinearity.

A second shortcoming of D2 based estimation concerns the use of intracranial depth electrodes for temporal lobe epilepsy. The invasive nature of the implanting depth electrodes greatly limits the diagnostic and treatment potential of the D2 method of seizure prediction. A more preferably prediction method would be compatible with convention, non-invasive EEG scalp recordings.

A final shortcoming of D2 based epileptic seizure prediction models is that computation of nonlinear dynamic algorithms takes several hours for just 15 minutes of EEG data. This shortcoming raises serious questions as to whether nonlinear dynamic algorithms can be optimized for real-time seizure prediction.

Accordingly, there is a need for an epileptic seizure detection and prediction system and method that is insulated from potential errors, is non-invasive, and can provide real-time seizure prediction on the order of minutes.

### SUMMARY OF THE INVENTION

The present invention answers this need by providing a system and method of seizure detection and prediction though the characterization of EEG recording data on the basis of “self-similar” structure.

An aspect of the present invention is an epileptic seizure detection and prediction method in which a scaling exponent characterizing self-similar structure is computed from the electrical patterns data of a patient's brain waves. In the preferred embodiment, non-invasive electroencephalogram recording data is used to compute the scaling exponent and related parameters, normalized c_{f} and fractal fraction, characterizing self-similar structure.

An object of the present invention is to provide a prediction/detection notification alert when the computed scaling exponent satisfies preselected parameters, such as a sustained scaling exponent value or rapid change in the scaling exponent value for a specified time interval.

A further aspect of the present invention includes rendering of a seizure detection alert when said scaling exponent value rapidly decreases from a value >1 then to a value <1 over a predetermined time interval.

Another aspect of the present invention includes rendering of a seizure detection alert when said normalized C_{f} decreases to a value of zero or said fractal fraction decreases to a value of 10⁻⁵.

Another object of the present invention includes rendering of a prediction warning for a forthcoming seizure when said scaling exponent value remains >1 for a predetermined time interval.

A further aspect of the present invention includes a system for real-time seizure warning comprising means for receiving electrical patterns data of a patient's brain waves, calculating means for computing a scaling exponent, normalized C_{f} and fractal fraction from the electrical patterns data, and warning means to render a notification of seizure prediction/detection based on the computed value of the scaling exponent, normalized C_{f} and fractal fraction.

A further aspect of the present invention is an epileptic seizure detection and prediction method in which a scaling exponent value is computed from the electrical patterns data of a patient's brain waves. In one embodiment, non-invasive electroencephalogram recording data is used to compute the scaling exponent value. In this embodiment, a sharp decrease in the scaling exponent value indicates seizure onset, and seizure duration is indicated by a subsequent rise and then decrease in the scaling exponent.

Another aspect of the present invention is an epileptic seizure detection and prediction method in which normalized C_{f} and fractal fraction values are computed from the electrical patterns data of a patient's brain waves.

### BRIEF DESCRIPTION OF THE DRAWINGS

FIG. 1 depicts the international 10/20 configuration of electrodes used to obtain scalp EEG recording data in the present invention.

FIG. 2 is a graphical plot of a scaling exponent value vs. time for a non-seizure patient.

FIG. 3 is a graphical plot of the calculated scaling exponent value vs. time for a non-seizure patient.

FIG. 4 is a comparative graphical plot of (top) EEG recording data vs. time and (bottom) calculated scaling exponent value vs. time for a patient experiencing seizure.

FIG. 5 is graphical plots of calculated scaling exponent value vs. time for three patients experiencing seizures.

FIG. 6 is a comparative graphical plot of seizure onset time determined by calculated scaling exponent values vs. seizure onset time determined by clinical means.

FIG. 7 is graphical plots of calculated scaling exponent value vs. time for six non-seizure patients.

FIG. 8 is comparative graphical plots of EEG recording data vs. time and calculated scaling exponent values vs. time and scaling behavior values vs. time for two patients experiencing seizures.

FIG. 9 is graphical plots (A) of seizure onset determined by calculated scaling behavior value detection methods vs. seizure onset time determined by clinical methods and (B) seizure onset time determined by calculated scaling exponent detection method vs. seizure onset time determined by clinical methods.

FIG. 10 is a graphical plot (A) of normalized scaling behavior values vs. time for a patient experiencing a seizure and (B) plot of estimated seizure duration determined from normalized scaling behavior values vs. clinically determined seizure duration times.

FIG. 11 is a (C) graphical plot of calculated scaling exponent values vs. time for a patient experiencing a seizure and (D) a plot of seizure duration time determined from calculated scaling exponent values vs. seizure duration time determined by clinical methods.

FIG. 12 is a graphical plot of calculated scaling exponent values vs. time for four patients experiencing seizures.

FIG. 13 is six graphical plots of the time evolution of the fractal fraction computed from an EEG scalp recording at different electrode locations.

FIG. 14 is two graphical plots of the time evolution of the fractal fraction computed from an EEG scalp recording at different electrode locations.

FIG. 15 is a schematic diagram of seizure evolution based on the calculation of fractal fraction values at EEG electrodes at four consecutive time intervals.

FIG. 16 is four graphical plots of the time evolution of the fractal fraction computed from an EEG scalp recording at different electrode locations.

FIG. 17 is four graphical plots of the time evolution of the fractal fraction computed from an EEG scalp recording at different electrode locations.

FIG. 18 is four graphical plots of the time evolution of the fractal fraction computed from an EEG scalp recording at different electrode locations.

FIG. 19 is a schematic diagram of seizure evolution based on the calculation of fractal fraction values at EEG electrodes at four consecutive time intervals.

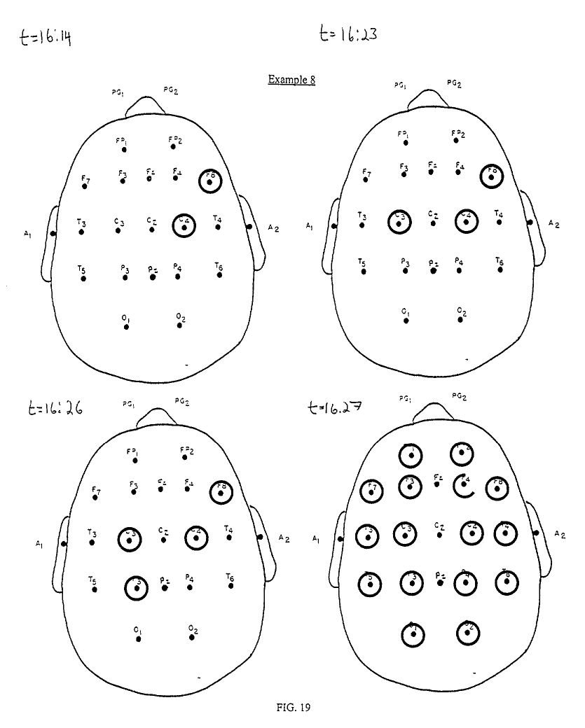

### DETAILED DESCRIPTION OF THE INVENTION

The present invention avoids the shortcomings of epileptic seizure prediction that utilize nonlinear dynamic methods by providing a system and method for epileptic seizure detection and prediction that characterizes electrical patterns data from brain waves based on evaluating self-similar “scaling” structure.

“Self-similarity” involves time series that appear to have similar shapes when plotted on different time scales. Time series, X(t), with self-similar scaling structure have power spectra, fₓ(ν), with a power-law frequency dependence, c_{f}|ν|^{-α}. Scaling structure in time series is common in biology, engineering, physics, and economics, and is typically called “colored noise.”

Typically, self-similar scaling structure in time series is characterized by nonparametic power spectrum estimates based on periodgrams or parametric maximum likelihood-based autoregressive (AR) modeling techniques. Periodgram based methods suffer from well-known problems of significant bias and high variance. Maximum likelihood-based methods such as the Whittle, Aggregated Whittle, and Local Whittle Methods, exhibit the least bias and variance, but at the cost of requiring long time series records and consuming significant amount of computation time. These drawbacks have precluded the real-time self-similar scaling structure characterization of EEG time series.

Developed for characterization of scaling structure in the analysis of telecommunications traffic in high digital speed networks, the Veitch-Abry algorithm,

fₓ(ν)˜c_{f}|ν|^{-α}

overcomes these drawbacks by allowing scaling structure characterization that is computationally efficient and statistically robust. Veitch-Abry algorithm's characterization of a time-series' scaling structure is based on the wavelet transform properties that reduces long range correlations in the time series to short range correlations in the transformed time-scale representation.

In one embodiment, the method of the present invention adapts the Veitch-Abry algorithm to provide for automated, real-time seizure detection, characterization and prediction from clinical scalp EEG recordings in human epileptic patients. The present invention evaluates hidden mathematical patterns in EEG recordings corresponding to “self-similar” or “fractal” structure. “Self-similar” and “fractal” refer to EEG signals that appear to have a similar shape when plotted on different time scales or magnifications.

In the present invention, the Veitch-Abry algorithm characterizes scaling structure by jointly estimating the scaling parameters α and c_{f}.

The scaling exponent, α, thus has important implications for the interpretation of EEG times series. First, any α value demonstrates the presence of scaling in a time series. Second, α provides an estimate of the power law characterizing the time-series' power spectrum, fₓ(ν). Third, α provides information about how previous EEG signal amplitudes are likely to affect the present EEG amplitudes.

c_{f} provides information about the overall amount of self-similarity present in the EEG recording. Higher c_{f} values indicate a greater proportion of the signal is displaying long-range dependent (LRD) behavior reflects large-scale neuronal synchronization. Consequently, c_{f} is useful in detecting seizures.

Fractal fraction provides information about what fraction of total EEG signal energy over a specified time interval is self-similar. Fractal fraction is derived from the Veitch-Abry algorithm by the following relation:

$$FractalFraction ( t ) = \frac{{\int }_{Flow}^{Fhtgh} {c}_{f} {\vert υ \vert}^{- a}}{{\int }_{Flow}^{Fhtgh} FFT ( x ( t ) )}$$

Here FFT(x(t)) is the Fourier Transform of a 256 point EEG time series record, and Fhigh and Flow represent the high and low frequency values of the EEG times series power spectra.

In the present invention, α, normalized c_{f} and fractal fraction parameters were computed to detect and predict seizures. The presence of self-similarity in an EEG recording can be characterized by “scaling exponent” α and measures of the proportion of the signal involved in scaling behavior, normalized c_{f} and fractal fraction. In the present invention, α values >1 indicate how previous EEG signal amplitudes are likely to affect the present EEG amplitudes. The higher the α value, the greater this influence. Consequently, α is useful in predicting seizures.

In an embodiment of the present invention, digital EEG records were preferably broken into consecutive blocks of 256 samples. Telefactor CTE 64 files were sampled at 200 Hz, so each block is 1.28 seconds in duration. Bio-logic CeeGraph V5 files were sampled at 256 Hz, so each block is 1 second in duration. Each block was prefiltered to reduce errors in discrete wavelet transform (DWT). Subsequently, a DWT was computed on each prefiltered block using the Daubechies compactly support wavelets. With 256 samples, the DWT produces a maximum of 8 scales. For each scale DWT coefficients were squared and averaged. Log₂ of each scale's average were calculated and corrected for nonlinearities.

Corrected log averages were plotted as a function of scale number. A straight line on the logscale plot indicates scaling structure, and estimates of a are derived from the plot Veitch-Abry algorithm. The α values were computed using the Veitch-Abry based on publicly available MatLab code by Veitch & Abry modified for use in the present invention. The modified MatLab code is provided in Appendix 1.

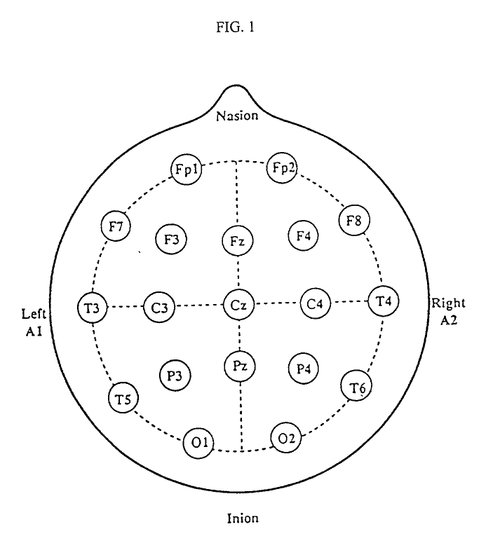

Referring to FIG. 1, the international 10/20 configuration of electrodes was used to obtain scalp EEG recording data from patients.

EEG data collection includes data collection from one or more of the following machines: 64 channel Telefactor Beehive, a 128 channel Telefactor Beehive, a portable 32 channel Telefactor Beehive 7, a 27 channel Bio-logic Sleepscan Traveler ambulatory recorder, a Bio-logic 64, and/or a 128 channel Ceegraph IV. Those skilled in the art will appreciate that other digital machines and a reader station may be used in the present invention. In one embodiment, the reader station transcribes the digital EEG proprietary file formats into ASCII for analysis.

Numerical computation is provided by a computer, such as a Pentium III class PC with C compiler and assembler support. Numerical computation and simulation is supported by one or more of the following software components: Matlab v.5.3 & Signal Processing, High-Order Spectral Analysis and Wavelet Toolboxes, S-Plus 2000; S-Plus Wavelets; S-Plus Spatial Stats; Labview v. 4.1 & Joint Time-Frequency Analysis and Wavelet and Filter Design Toolkits.

To determine the efficacy of detecting and predicting seizures based on α values, time-dependent α values were computed from EEGs in patients with and without ongoing seizures.

In Examples 1 and 2, α values were determined in patients without ongoing seizures.

### EXAMPLE 1

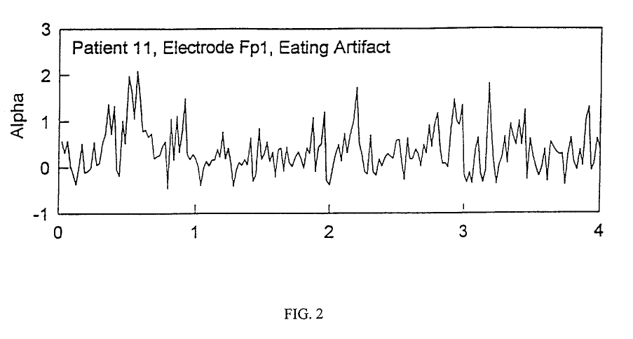

Non-seizure EEG recording data was collected from among 15 patients, specifically Patient 11 at electrode Fp1 (eating artifact), using the international 10/20 configuration of electrodes. Referring to FIG. 2, α values were calculated and plotted for a 4 minute time interval.

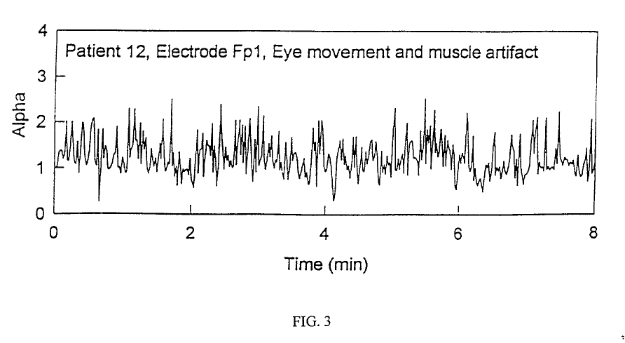

Similarly, EEG recording data was collected from Patient 11 at electrode Fp1 (eye movement and muscle artifact). Referring to FIG. 3, α values were calculated and plotted for an 8 minute time interval.

The results of FIGS. 2 and 3 demonstrate a random fluctuation in α values in non-seizure EEGs.

### EXAMPLE 2

Among a test group of 25 epileptic patients, non-seizure EEG recordings containing a variety of common artifacts imitating seizures were collected using the international 10/20 configuration of electrodes.

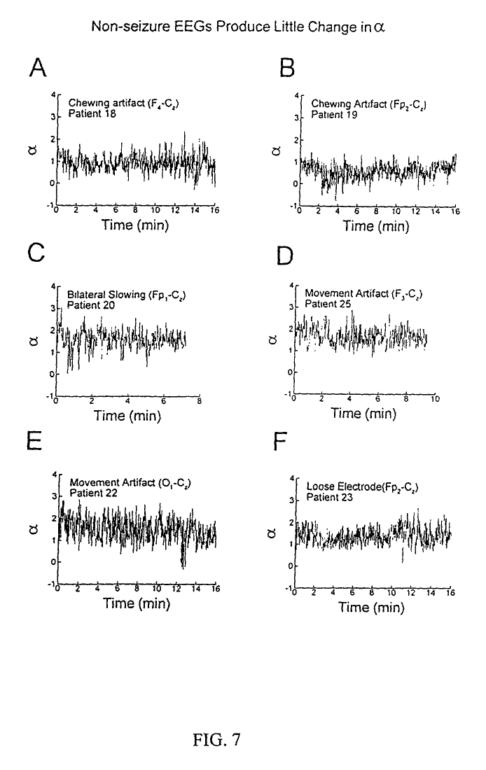

Referring to FIG. 7, a values were calculated and plotted over a time interval for 6 non-seizure patients. Patient 18 EEG recording data was collected from electrode pair F₄-C_{z} (chewing artifact), and α values were calculated and plotted for a 16 minute time interval in graph A. Patient 19 EEG recording data was collected from electrode pair Fp₂-C_{z} (chewing artifact), and α values were calculated and plotted for a 16 minute time interval in graph B. Patient 20 EEG recording data was collected from electrode pair Fp₁-C_{z} (bilateral slowing), and α values were calculated and plotted for an 8 minute time interval in graph C. Patient 25 EEG recording data was collected from electrode pair F₃-C_{z} (movement artifact), and a values were calculated and plotted for a 10 minute time interval in graph D. Patient 22 EEG recording data was collected from electrode pair O₁-C_{z} (movement artifact), and α values were calculated and plotted for a 16 minute time interval in graph E. Patient 23 EEG recording data was collected from electrode pair Fp₂-C_{z} (loose electrode), and a values were calculated and plotted for a 16 minute time interval in graph F.

Plots A-F show random fluctuation, and the absence of systemic rises and dips, in α values.

In Examples 3 through 5 the time evolution of α values in patients experiencing seizures was determined.

### EXAMPLE 3

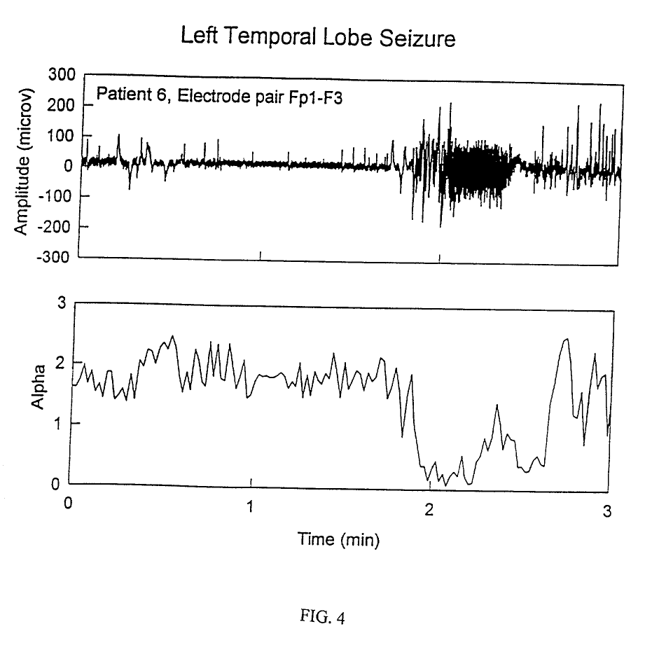

Among 15 patients, EEG recording data from Patient 6 was collected at electrode pair Fp1-F3 (left temporal lobe seizure). Referring to FIG. 4, the top graph shows the electrographic record of a left temporal lobe seizure with conventional scalp electrodes. The time evolution of α values were computed for only the F3 electrode, and plotted for a time interval of 3 minutes, shown in the bottom graph.

It is noted that a values computed from the Fp1 electrode provide a similar, steep dip in α. Typically, α values computed from referential electrodes yield more information than α values computed from bipolar pairs. This result suggests that the scaling structure contained the referential electrodes is common since subtraction cancels much of the scaling. The characteristic decrease in α values to <1 that accompanies the seizure and persists after the high voltage EEG activity decreases is clearly visible. This steep, sustained decrease in the time evolution of α is characteristic of EEGs containing seizures. This observation is different in the present example in that the seizure detection in the present invention was derived from scalp EEG recordings and show resistance to many different kinds of artifacts that sometimes plague clinical recordings. In addition, the Veitch-Abry algorithm allows for quick calculation and real time implementation.

### EXAMPLE 4

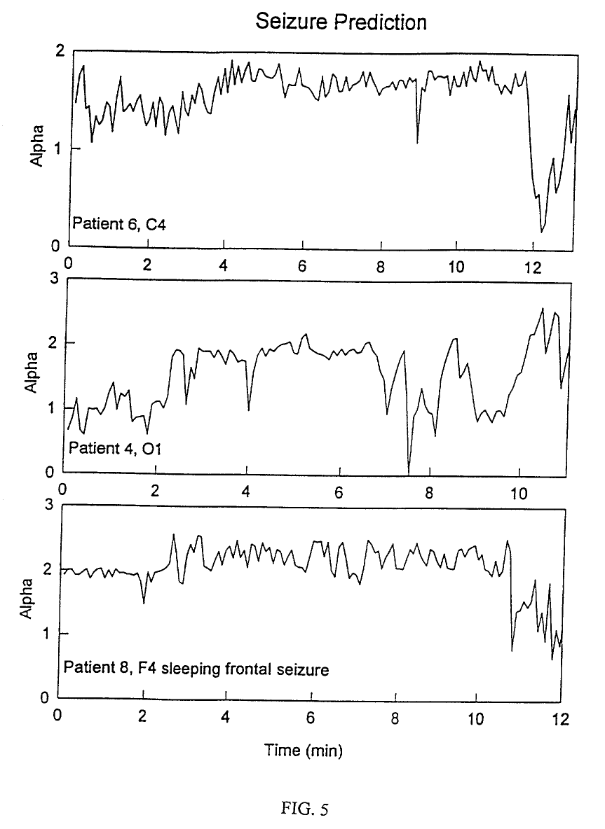

Referring to FIG. 5, α values for three patients among a group of 15 patients experiencing seizures were plotted over time intervals.

The top graph depicts the time evolution of a values for Patient 6, EEG recording data collected at electrode C4, for 13 minutes. The graph shows a sustained α>1 for approximately 10 minutes followed by a rapid decrease in α to <1 during the seizure (between the 11 minute and 12 minute interval).

The middle graph depicts the time evolution of α values for Patient 4, EEG recording data collected at electrode O1, for 11 minutes. The graph shows a sustained α>1 for approximately 4-5 minutes (from about the 2 minute to about the 7 minute interval) followed by a rapid decrease in α to <1 during the seizure (between the 6 minute and the 8 minute interval).

The bottom graph depicts the time evolution of α values for Patient 8 (sleeping), EEG recording data collected at electrode F4, for 12 minutes. The graph shows a sustained α>1 for approximately 10 minutes followed by a rapid decrease in α to <1 during the seizure (between the 10 minute and the 12 minute interval).

These results demonstrate not only detection of seizure based on α values, but establish a stereotyped pre-seizure time interval characterized by a sustained rise in the time evolution of α, i.e., α>1.

### EXAMPLE 5

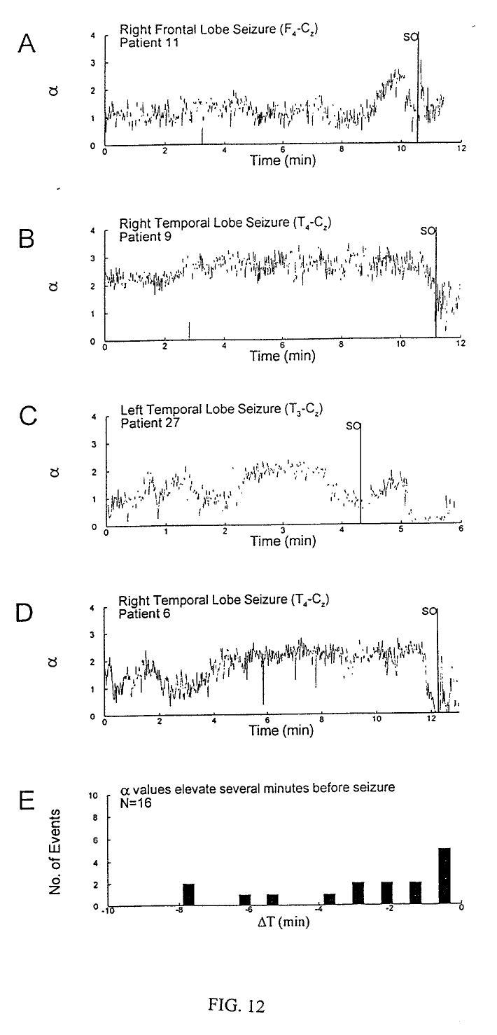

Referring to FIG. 12, Graphs A-D, time evolution of α values for 4 patients experiencing seizures is plotted.

Graph A shows α values calculated for patient 11 (right frontal lobe seizure), from EEG recording data at electrode pair F₄-C_{z}, and plotted against a 12 minute time interval. Clinically determined seizure onset (SO) is designated by a solid vertical line.

Graph B shows α values calculated for patient 9 (right temporal lobe seizure), from EEG recording data at electrode pair T₄-C_{z}, and plotted against a 12 minute time interval. Clinically determined seizure onset (SO) is designated by a solid vertical line.

Graph C shows α values calculated for patient 27 (left temporal lobe seizure), from EEG recording data at electrode pair T₃-C_{z}, and plotted against a 6 minute time interval. Clinically determined seizure onset (SO) is designated by a solid vertical line.

Graph D shows α values calculated for patient 6 (right temporal lobe seizure), from EEG recording data at electrode pair T₄-C_{z}, and plotted against a 13 minute time interval. Clinically determined seizure onset (SO) is designated by a solid vertical line.

The results of Graphs A-D indicate that α values ranging from 1 to 4 indicate a high persistence where high amplitude EEGs are likely. α values form 0 to 1 indicate a long range dependence in the form of a periodic discharge. Thus, a pre-seizure state is indicated by a rise in α values, while seizure onset is indicated by a dip in α values, followed by a rapid rise as a seizure develops.

Referring to FIG. 12, Graph E, the results of 16 out of 22 seizures indicates that there is a pre-seizure interval of several minutes based on the pattern of α results.

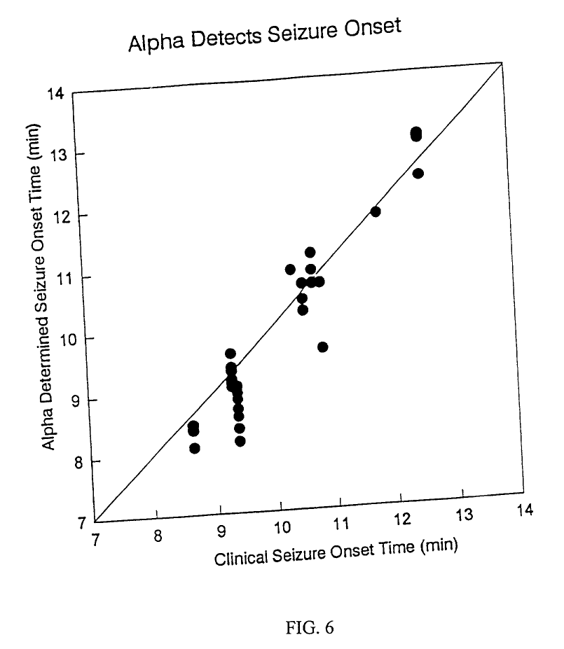

Referring to FIG. 6, the time evolution of a provides reasonable estimates of the seizure onset time. Data from 9 patients were used to compare α-determined seizure onset with clinically determined seizure onset. Each dot represents a pair of α and clinically determined seizure onset times. Multiple dots with a single clinical onset time represent slight differences between α onset times in different referential electrode in a single patient. The solid line represents an ideal correlation between the clinical seizure onset time and the seizure onset time determined by the steep decrease in α. The graph shows excellent agreement between α-determined and clinically determined methods.

Based on the data of FIG. 6, seizure onset time and duration were compared between the automated and clinical methods through a paired test for the difference in means for repeated measures data. Correlational analyses in the presence of repeated measurements were performed to evaluate the relationship between the automated and clinical methods with respect to onset time and duration of seizure. The statistics are provided in Table 1.

TABLE 1

| | Automated α - Clinical Average Difference |
|---|---|
| Response | in Seconds (Standard Error) |
| Onset Time | −4.75 (4.35) |
| Duration | 27.65 (8.18) |

Estimates of correlation produce a concordance correlation coefficient of 0.876 (standard error=0.081) for clinical vs. automated onset times, and an estimate of 0.454 (standard error=0.226 for clinical vs. automated duration times.

Referring to FIG. 7, the average pre-seizure interval across all patients was 4.21 minutes with highest pre-seizure interval of 9.77 minutes and a lowest of 0.79 minutes.

In Example 6 both α and c_{f} values were calculated and plotted to determine stereotypical changes in the respective values for detection of seizures.

### EXAMPLE 6

Among 25 patients, EEG recording data from 2 patients was collected using the 10/20 system.

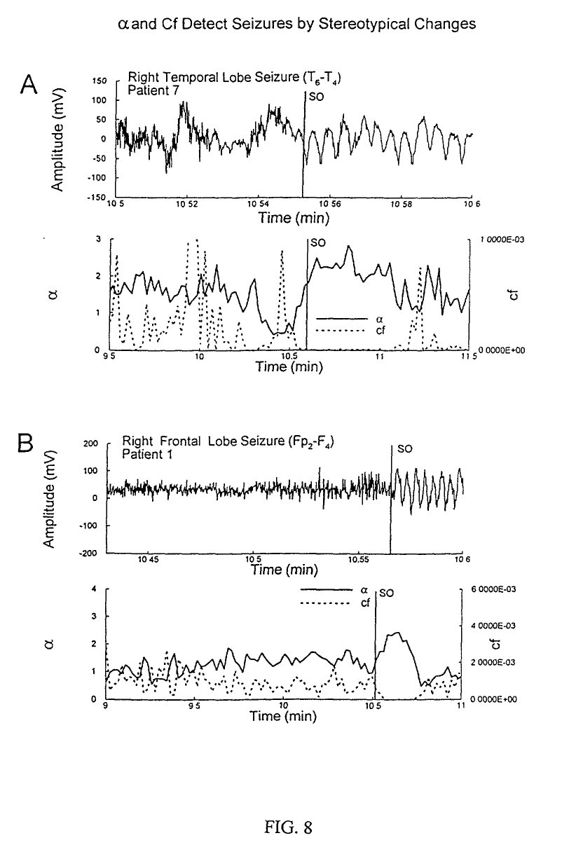

Referring to FIG. 8, plot A (top) provides EEG recording data of Patient 7 with conventional surface electrodes at temporal locations T₆ and T₄ . Solid vertical line (SO) represents the clinical onset of a right temporal lobe seizure as determined by expert visual analysis. Plot A (bottom) depicts α (horizontal solid line) and c_{f} (dashed line) values calculated from an overlapping segment of the EEG record. Each point in α and c_{f} profiles corresponds to the analysis of 256 times samples in the original EEG record. The vertical SO line indicates seizure onset as above.

Referring to FIG. 8, plot B (top) provides EEG recording data of Patient 1 with conventional surface electrodes at temporal locations Fp₂ and F₄ Solid vertical line (SO) represents the clinical onset of a right frontal lobe seizure as determined by expert visual analysis. Plot B (bottom) depicts α (horizontal solid line) and c_{f}(dashed line) values calculated from an overlapping segment of the EEG record. Each point in α and c_{f} profiles corresponds to the analysis of 256 times samples in the original EEG record. The vertical SO line indicates seizure onset as above.

From the standpoint of confirmation of seizure detection, the results of Example 6 demonstrate at seizure onset, a rapid increase in α and a rapid decrease in c_{f}. Specifically, at SO in Plot A (bottom) and Plot B (bottom) it is observed that α increases rapidly to above 2, and in c_{f} decreases rapidly to about 0. This general pattern of a rapid increase in α and a rapid decrease in c_{f} was observed in 22 of 25 (88%) EEG seizure records, demonstrating the high sensitivity of the detection method.

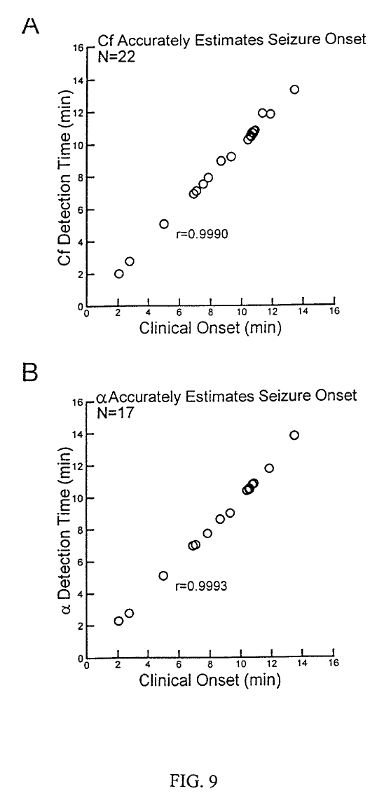

Referring to FIG. 9, Graph A, the high correlation between the clinically determined seizure onset times for temporal and frontal lobe seizure and stereotypic changes in c_{f} is shown. Sharp declines in c_{f} confirm seizure detection in 22 out of 25 records, r=0.999. In 3 records, no clear changes were observed.

Referring to FIG. 9, Graph B, sharp rises in a show a high correlation, 17 out of 25 records, r=0.9993, with clinically determined seizure onset times. In addition to the 3 records noted for no changes observed in c_{f}, 5 additional records showed no clear changes in α at the time of seizure onset.

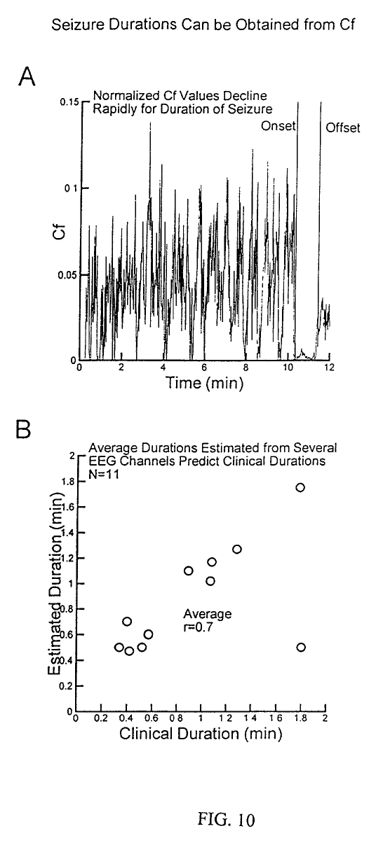

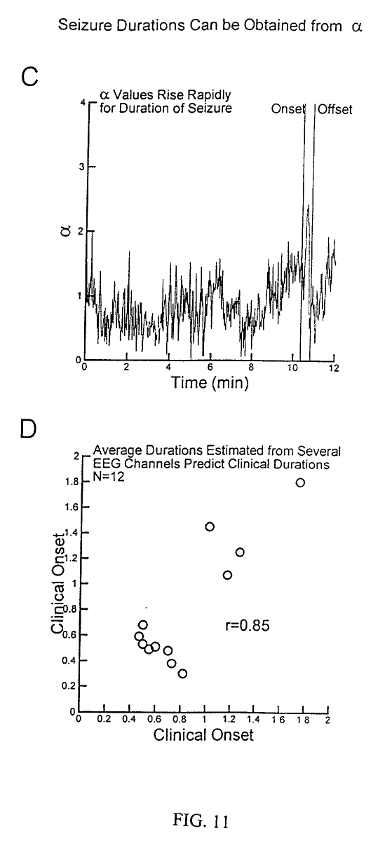

Referring to FIGS. 10 and 11, the values of α and c_{f} also provide an indicator of seizure duration.

Referring to FIG. 10, Plot A, normalization of c_{f} by the ongoing EEG signal amplitude variance and plotted against a time interval shows a sharp decline in c_{f} sustained for seizure duration. Referring to FIG. 10, Plot B, a comparison of clinically determined seizure durations estimated by normalized c_{f} shows a good correlation (11 out of 15 records).

Referring to FIG. 11, Plot C, α values plotted against a time interval during seizure show a marked rise which is sustained during the seizure. Referring to FIG. 11, Plot D, a strong correlation (12 out of 15 records) between clinically determined seizure duration and α-rise is indicated.

In an embodiment of the present invention, a non-invasive monitor is provided to record brain wave data. Preferably, EEG recording data is collected from a patient via an electrode.

Using a microprocessor, calculation software utilizing the modified Veitch-Abry algorithm is used to compute α and c_{f} values from the EEG recording data against predetermined time intervals. A notification module is provided with predetermined parameters based on one or both of the α, scaling exponent, and c_{f}, scaling behavior, values for a predetermined time interval. When said parameters satisfy a sustained value of α>1, a sharp decrease in α, a sharp increase in α, and/or a sharp decrease c_{f}, the notification of seizure is preferably provided to the patient or health care provider.

Examples 7 and 8 demonstrate that a drop in fractal fraction values further provides a good time evolution determiner of a seizure across the brain.

### EXAMPLE 7

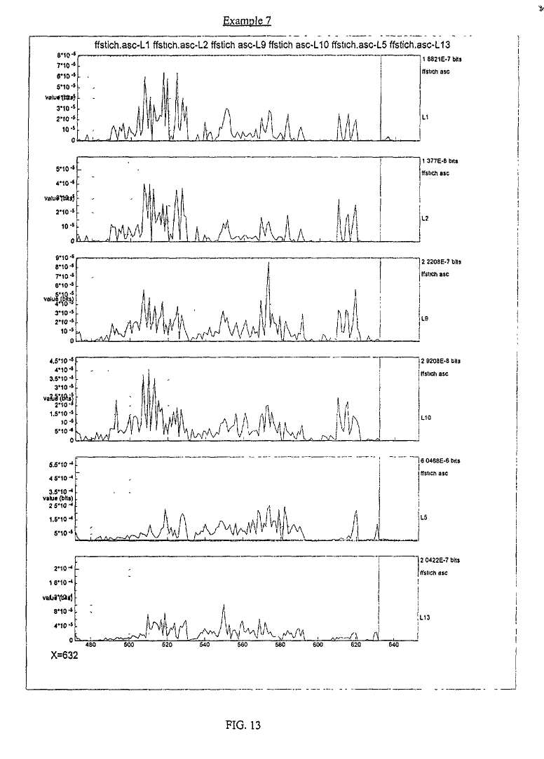

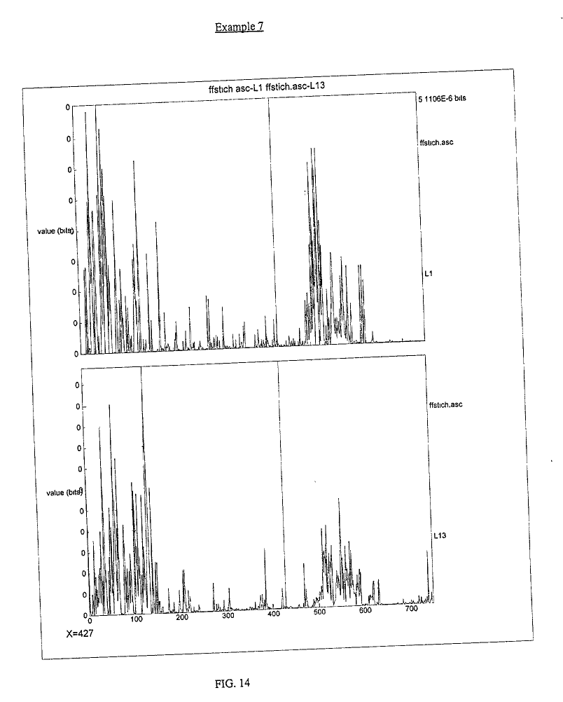

Referring to FIGS. 13-15, EEG recording data from a patient experiencing a seizure was collected using the 10/20 system. FIGS. 13 and 14 show the time evolution of the fractal fraction computed from EEG scalp recording data at different electrode locations. Each point of the fractal fraction corresponds to 256 EEG samples, corresponding to one second of time. Seizures are indicated by fractal fractions valves that fall below 10⁻⁵. As can be seen from the plots, the fractal fraction falls to the 10⁻⁵ valve at different times at different electrode locations.

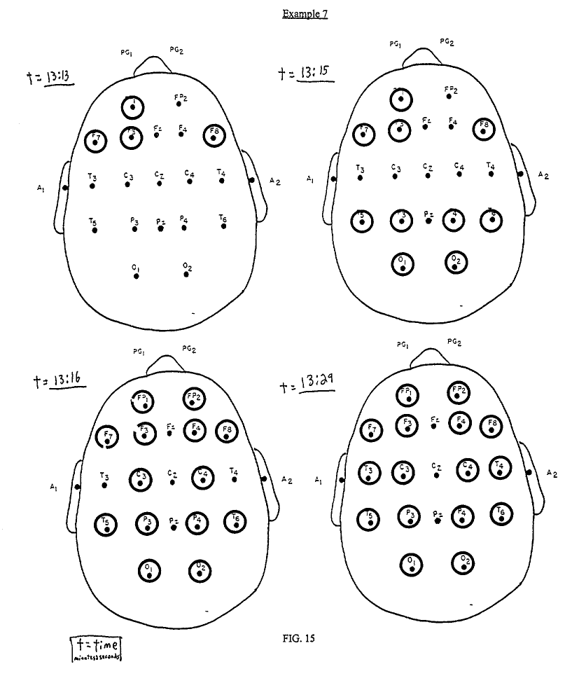

Referring to FIG. 15, a summary of seizure onset corresponding to a drop in the fractal fraction valve at particular electrode locations is summarized graphically. At time interval 13 minutes, 13 seconds, the fractal fraction value drops bilaterally, indicating the seizure does not have a single focus in the frontal region. At time interval 13 minutes, 15 seconds, the seizure spreads to the parietal and occipital regions. Within a second, at time interval 13 minutes, 16 seconds, the seizure rapidly spreads, and at time interval 13 minutes, 29 seconds, the seizure rapidly generalizes to encompass the entire cortex.

Traditional clinical diagnosis indicated the seizure origin was in left temporal lobe (T₃ -T₅). Although the left temporal lobe was removed, the patient still has seizures. The results of this example, however, suggest that given the bilateral frontal origin of the seizure a poor surgical outcome is indicated. This hypothesis was confirmed as subsequent surgical removal of the temporal lobe did not cure the patient's seizures.

### EXAMPLE 8

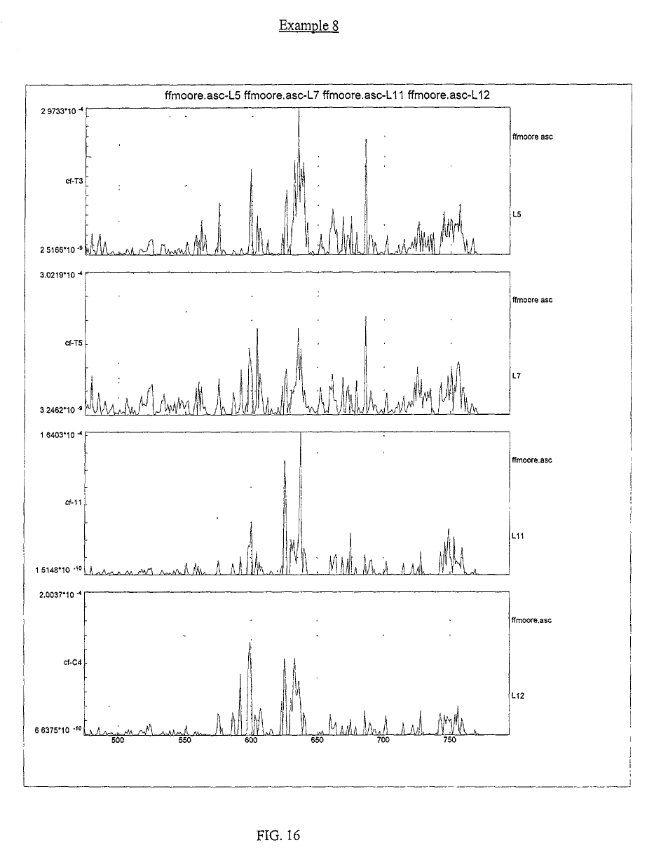

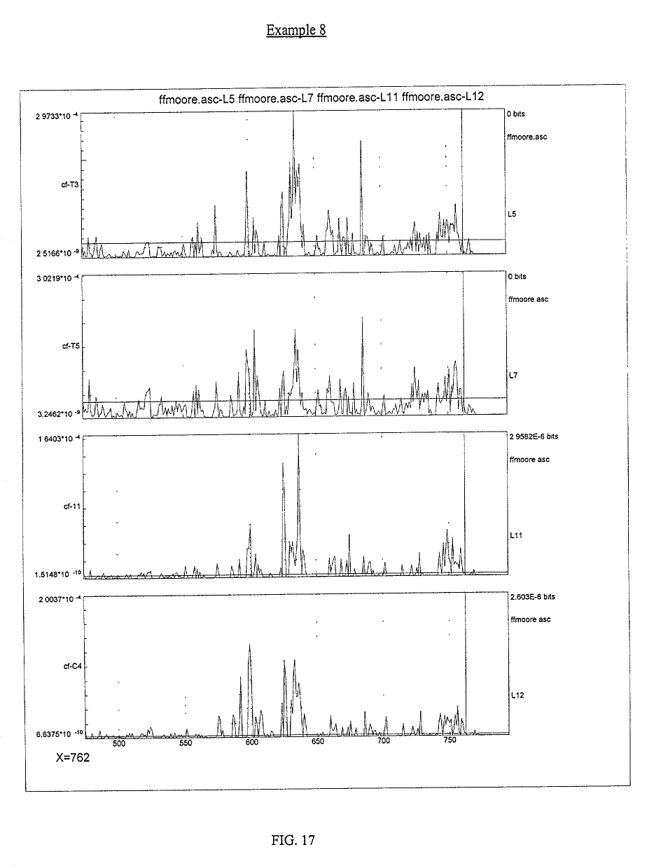

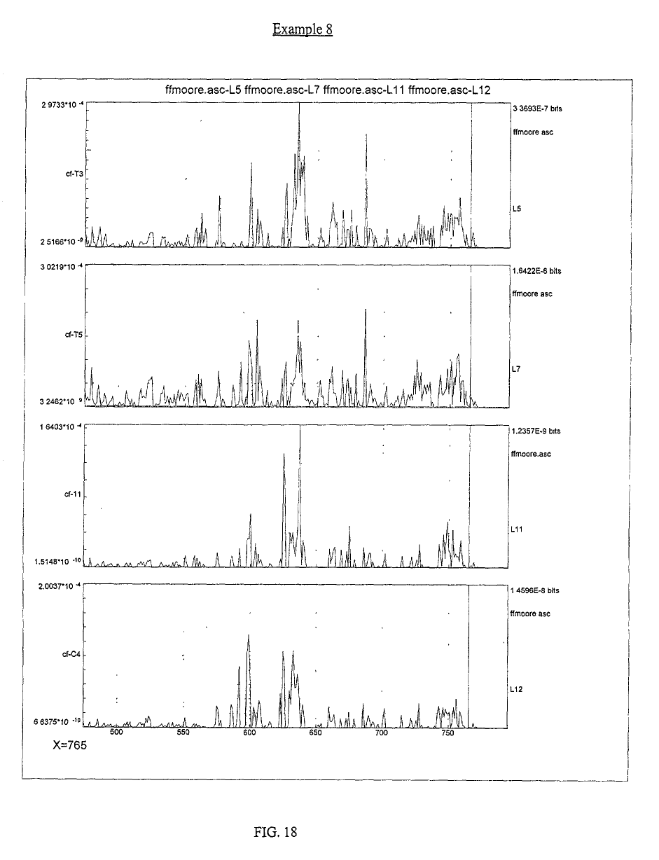

Referring to FIGS. 16-1, fractal fraction values were calculated from EEG recording data using the 10/20 system from a patient experiencing a seizure. Referring to FIGS. 16-18, the graphical plots show different seizure onset times at different electrode locations as indicated by a drop in the fractal fraction value to less than 10⁻⁵ at the respective different electrode locations.

At time interval 16 minutes, 14 seconds, the fractal fraction value drops laterally at electrodes C₄ and F₈ and spreads rapidly to encompass the entire cortex through a 13 second time interval. The results of the calculation of fractal fraction value indicates that, based on the seizure starting laterally, surgical removal of the right temporal lobe is indicated.

Removal of the right temporal lobe of the subject patient resulted in the patient becoming seizure free.

As will be apparent to one of ordinary skill in the art, the analysis of the α, c_{f} and fractal fraction values allows prediction of the onset of an epileptic seizure. By monitoring those values in real time, an alarm may be provided to alert both the patient and caregiver of the imminent onset of seizure. In the preferred embodiment, monitoring may be accomplished through MATLAB code calculations. Exemplary MATLAB code for α and c_{f} calculations is included in Appendix 1. Exemplary MATLAB code for fractal fractions calculation is included in Appendix 2. Through monitoring and notification, both patient and caregiver can provide for the needs of the patient associated with the epileptic seizure.

Accordingly, while the invention has been described with reference to the structures and methods disclosed, it is not confined to the details set forth but is intended to cover such modifications or changes as may fall within the scope of the following claims.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

What is claimed is:

1. A method for epileptic seizure warning comprising:
a) receiving electrical patterns data of a patient's brain waves;
b) computing a scaling exponent from the electrical patterns data; and
c) rendering a notification when said scaling exponent satisfies preselected parameters.

2. The method of claim 1 wherein said electrical patterns data includes electroencephalogram recording data.

3. The method of claim 2 wherein said preselected parameters include a scaling exponent value >1 sustained for a predetermined time interval.

4. The method of claim 3 wherein said time interval is greater than 1 second.

5. The method of claim 3 wherein said time interval is between 1 second and 10 minutes.

6. The method of claim 3 wherein said time interval is greater than 3 seconds.

7. The method of claim 3 wherein said time interval is greater than 10 seconds.

8. The method of claim 3 wherein said time interval is greater than 30 seconds.

9. The method of claim 3 wherein said time interval is greater than 1 minute.

10. The method of claim 3 wherein said time interval is greater than 2 minutes.

11. The method of claim 1 wherein said preselected parameters include a scaling exponent value >1 sustained for a predetermined time interval.

12. The method of claim 1 wherein said preselected parameters include a scaling exponent value >1 sustained for a first predetermined time interval and a subsequent decrease of the scaling exponent value to <1 during a second predetermined time interval.

13. The method of claim 12 wherein said first predetermined time interval is between 1 second and 10 minutes and said second predetermined time interval is less than 2 minutes.

14. The method of claim 13 wherein said second predetermined time interval is less than 1 minute.

15. The method of claim 14 wherein said second predetermined time interval is less than 30 seconds.

16. The method of claim 15 wherein said second predetermined time interval is less than 10 seconds.

17. The method of claim 16 wherein said second predetermined time interval is less than 5 seconds.

18. The method of claim 1 wherein said preselected parameters include a rapid rise in scaling exponent values to >2 over a predetermined time interval and a subsequent decrease of scaling behavior values to about 0 during said time interval.

19. A system for epileptic seizure warning comprising:
a) means for receiving electrical patterns data of a patient's brain waves;
b) calculating means for computing a scaling exponent from the electrical patterns data; and
c) warning means to provide a notification when said scaling exponent satisfies predetermined parameters.

20. The system of claim 18 wherein said means for receiving electrical patterns data includes an electroencephalogram recording.

21. The system of claim 19 wherein said calculating means is a scaling exponent calculation program running on a microprocessor.

22. The system of claim 18 wherein said predetermined parameters include a scaling exponent value of >1 sustained for a predetermined time interval.

23. The system of claim 18 wherein said preselected parameters include a scaling exponent value >1 sustained for a first predetermined time interval and a subsequent decrease of the scaling exponent value to <1 during a second predetermined time interval.

24. The system of claim 20 wherein said predetermined parameters include a scaling exponent value of >1 sustained for a predetermined time interval

25. The system of claim 20 wherein said preselected parameters include a scaling exponent value >1 sustained for a first predetermined time interval and a subsequent decrease of the scaling exponent value to <1 during a second predetermined time interval.

26. The system of claim 24 wherein said first predetermined time interval is between 1 second and 10 minutes and said second predetermined time interval is less than 2 minutes.

27. The method of claim 23 wherein said time interval is between 1 second and 10 minutes.

28. A method for treating a patient experiencing epileptic seizures comprising:
a) receiving electrical patterns data of a patient's brain waves;
b) computing scaling exponent values from the electrical patterns data for a predetermined time interval; and
c) determining a treatment regimen for the patient based on said scaling exponent values.

29. A method for treating a patient experiencing epileptic seizures comprising:
a) receiving electrical patterns data of a patient's brain waves;
b) computing scaling behavior values from the electrical patterns data for a predetermined time interval; and
c) determining a treatment regimen for the patient based on said scaling behavior values.

30. A method for treating a patient experiencing epileptic seizures comprising:
a) receiving electrical patterns data of a patient's brain waves;
b) computing fractal fraction values from the electrical patterns data for a predetermined time interval; and
c) determining a treatment regimen for the patient based on said fractal fraction values.

31. The method of claim 30 further comprising the step of mapping seizure evolution based on the fractal fraction values, prior to step (c).

32. A method for epileptic seizure warning comprising:
a) receiving electrical patterns data of a patient's brain waves;
b) computing a fractal fraction value from the electrical patterns data; and
c) rendering a notification when said fractal fraction value satisfies pre-selected parameters.

33. The method of claim 31 wherein said electrical patterns data includes electroencephalogram recording data.

34. The method of claim 32 wherein said pre-selected parameters include a fractal fraction value ≦10⁻⁵ for a predetermined length of time.
